(19) United States
(12) Patent Application Publication
(10) Pub. No.: US 2018/0268936 A1
(43) Pub. Date: Sep. 20, 2018

(54) PULSE OXIMETER USER INTERFACE CUSTOMIZED TO A DOCTOR

(71) Applicant: KONINKLIJKE PHILIPS N.V., EINDHOVEN (NL)

(72) Inventors: John CRONIN, BONITA SPRINGS, FL (US); Seth Melvin CRONIN, BONITA SPRINGS, FL (US)

(73) Assignee: KONINKLIJKE PHILIPS N.V., EINDHOVEN (NL)

(21) Appl. No.: 15/761,484

(22) PCT Filed: Sep. 28, 2016
(86) PCT No.: PCT/EP2016/073037

§ 371 Date: Mar. 20, 2018

Related U.S. Application Data
(60) Provisional application No. 62/233,502, filed on Sep. 28, 2015.

(30) Foreign Application Priority Data
Mar. 18, 2016 (EP) 16161273.4

(51) Int. Cl. G16H 50/20; A61B 5/1455; A61B 5/00; A61B 5/145; G16H 40/63; G16H 50/70

(52) U.S. Cl. CPC G16H 50/20; A61B 5/14551; A61B 5/7275; A61B 5/742; A61B 5/021; A61B 5/0022; A61B 5/7445; G16H 40/63; G16H 50/70; A61B 5/14539

(57) ABSTRACT

Systems and methods for predicting a physiological condition and customizing displayed physiological data. A pulse oximeter and second physiological sensor are connected to a control unit that controls a patient monitor display. A medical professional uses the patient monitor display to upload a list of symptoms and data from the pulse oximeter and the second physiological sensor to a cloud-based prediction engine to determine a likely physiological condition and determine key physiological indicators associated with the physiological condition that was not uploaded. A plurality of analysis and customization tools are also provided for analyzing, correlating, tagging, labeling, and annotating the displayed data.

### BACKGROUND OF THE INVENTION

Pulse oximetry is an effective and non-invasive method of monitoring of and acquiring oxygen saturation (SpO₂) level and perfusion index of a patient. It is very useful in many situations where monitoring a patient's oxygenation level is important. For example, pulse oximetry is useful in emergency care situations, surgery, post-anesthetic care, and monitoring oxygenation of newly-born infants. Based on the acquired pulse oximetry data, the medical personnel assesses the patient's pulse oximeter data to determine the patient's health status.

In determining the patient's health status, the medical professional may also analyze other physiological data in combination with the pulse oximetry data. The combination of multiple physiological data may be fed to a prediction software to determine an accurate and likely physiological condition. It is also useful to determine the physiological indicators associated with a condition and suggests to a medical professional to measure all the physiological indicators. Such a system will prevent the medical professional from overlooking key indicators for the patient's physiological condition. In addition, there is also a need to provide the medical professional a suite of analytics and customization tools to customize the data obtained from the patient monitors.

### SUMMARY OF THE CLAIMED INVENTION

Embodiments of the present invention relates to systems and method for predicting the condition of a patient and customizing displayed physiological data. The system comprises a pulse oximeter, a plurality of physiological sensors, a control unit, and a patient monitor for displaying and customizing data. The system allows a medical professional to upload pulse oximeter data, physiological sensor data, and a list of symptoms to a cloud-based prediction engine to determine a likely physiological condition. After determining a likely physiological condition, the physiological indicators associated with the physiological condition are determined so that physiological indicators not uploaded by the medical professional are suggested for measurement or uploading. The patient monitor also allows a user to analyze and customize data using data analysis and customization tools.

### BRIEF DESCRIPTION OF THE DRAWINGS

The accompanying drawings, which are included to provide a further understanding of the invention, are incorporated herein to illustrate embodiments of the present invention. Along with the description, they also serve to explain the principle of the invention. In the drawings:

FIG. 1 illustrates a system of the preferred embodiment of the present invention.

FIG. 2 illustrates a preferred embodiment of a method of the present invention.

FIG. 3 illustrates an exemplary flow diagram of the display software in accordance with an embodiment.

FIG. 4 illustrates an exemplary flow diagram of the suggestion module in accordance with an embodiment.

FIG. 5 illustrates various sections displayed on the patient monitor in accordance with some embodiments.

FIG. 6 shows still another system according to some embodiments.

FIG. 7 shows various components of the patient monitor according to some embodiments of the present invention.

FIG. 8A-8D illustrates various GUIs in accordance with an embodiment of the present invention.

FIG. 9 illustrates a flow diagram of a new data tag tool in accordance with an embodiment of the present invention.

FIG. 10 illustrates a flow diagram of a search data tag tool in accordance with an embodiment of the present invention.

FIG. 11 illustrates a flow diagram of a correlate variable tool in accordance with an embodiment of the present invention.

FIG. 12 illustrates a flow diagram of a download additional tool in accordance with an embodiment of the present invention.

### DETAILED DESCRIPTION OF THE EMBODIMENTS

Embodiments of the present invention relate to a method for predicting a condition of the patient and customizing the display of a patient monitor comprising: acquiring pulse oximeter data using a pulse oximeter and second physiological sensor data using a second sensor; uploading symptoms, the acquired pulse oximeter data, and the acquired second physiological sensor data to a cloud-based prediction engine; predicting using the cloud-based prediction engine a physiological condition by matching the uploaded symptoms, the acquired pulse oximeter data, and the acquired second physiological sensor data to a cloud database; suggesting by the cloud-based prediction engine a measurement of a third physiological sensor data associated with the predicted physiological condition; acquiring the third physiological sensor data associated with the physiological condition; displaying the acquired pulse oximeter data, the acquired second physiological sensor data, the predicted physiological condition, and the third physiological sensor data; and customizing the displayed data using a data analysis and customization tool.

The system comprises a pulse oximeter, a second physiological sensor, a third physiological sensor, a cloud-based prediction engine, and a patient monitor with a display, a user interface, a processor, a communication module and a memory module with a data analysis and customization tool.

The pulse oximeter is preferably a portable pulse oximeter device adapted to be worn on a patient's finger and adapted to measure the oxygen saturation of the patient. Alternatively, the pulse oximeter is adapted to be attached onto the patient's ear, toe, or a body part other than the patient's finger.

In a preferred embodiment of the present invention, a doctor connects a pulse oximeter to a patient. On the user interface of a patient monitor, the doctor inputs the symptoms being experienced by the patient. The doctor also selects other physiological sensors connected to the patient, such as a temperature sensor and skin conductance sensors. The inputted symptoms, the pulse oximeter data and the data from the selected sensors are uploaded to a cloud-based prediction engine to determine a likely physiological condition, for example, asthma. It is then determined that breathing rate is an important physiological indicator for asthma, so the measurement of breathing rate is suggested and displayed on the patient monitor. Afterwards, the doctor accesses a data customization tool on the patient monitor. The doctor displays a plethysmograph waveform, selects certain points on the waveform and adds labels such as “took medication,” “reported headache,” or “went to the restroom.” The doctor further downloads a correlation tool from a medical data customization network to perform a correlation of different physiological data.

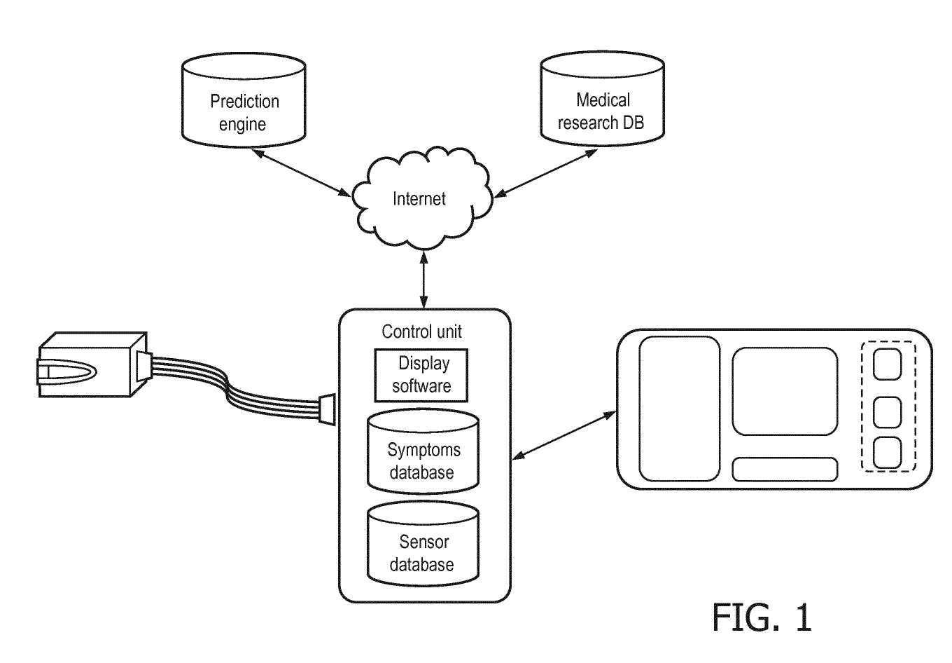

FIG. 1 illustrates an exemplary system of the present application according to some embodiments. A pulse oximeter 102 is connected, e.g., via an integrated cable connector 104 to a control unit 106 having a display software 108, a symptoms database 110, and a sensor database 112. The control unit 106 connects to the internet 114 to access a prediction engine 116 and a medical research database 118. The control unit 106 is also coupled with a patient monitor 120 with a user interface displaying multiple sections corresponding to, for example, a sensor data section 122, a symptoms section 124, an available sensors section 126, and a suggested sensor section 128. The control unit 106 is a computing device capable of receiving and storing data from a plurality of sensors, communicating to an external network, processing data, and controlling peripheral devices, such as a display device.

The cloud-based prediction engine 116 of the present invention refers to a cloud-based software that receives data and outputs a predicted physiological condition. A cloud-based prediction engine 116 receives data uploaded by a user and uses these data together with data from other sources to determine a likely physiological condition. Other sources of data may include historical data from the same user, statistical data from a population of patients, and published medical data by researchers, medical institutions, and medical companies. Prediction models may include those described in U.S. Pat. App. No. 2014/0344208. The medical research database 118 in accordance with an embodiment of the present invention is an online database of medical research and publications relating to physiological conditions and their associated symptoms and physiological indicators.

The patient monitor 120 of the present invention is a display-capable device with a user interface for receiving inputs from a user and outputting visual information, for example. The graphical user interface (GUI) of the patient monitor 120 is subdivided into a plurality of sections corresponding to different categories of information.

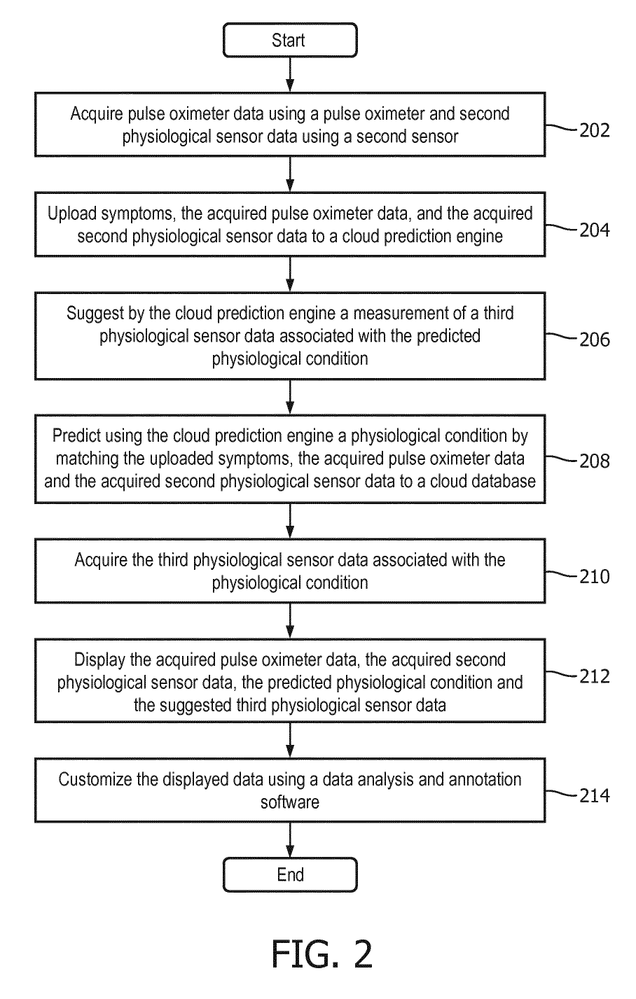

Illustrated in FIG. 2 is a flow diagram of the preferred method of the present invention. A pulse oximeter (i.e., a first sensor) and a second sensor is attached to the patient to acquire pulse oximeter data and second physiological sensor data, respectively (step 202). A list of symptoms is inputted into a user interface and the list of symptoms, along with the acquired pulse oximeter data, and the acquired second physiological data are then uploaded to a cloud-based prediction engine (step 204). The cloud-based prediction engine then proceeds to compare the uploaded data to data in a cloud-based database to predict a physiological condition (step 206). The physiological indicators associated with the predicted physiological condition are identified and a third physiological data associated with the predicted physiological condition and which was not previously uploaded by the user is suggested to the user for measurement (step 208). The user then proceeds to acquire from the patient the third physiological data associated with the predicted physiological condition (step 210). On a display device on the patient monitor, the acquired pulse oximeter data, the acquired second physiological data, the predicted physiological condition, and the acquired third physiological data are displayed (step 212). Afterwards, the user customizes the displayed data using a data analysis and customization tool (step 214).

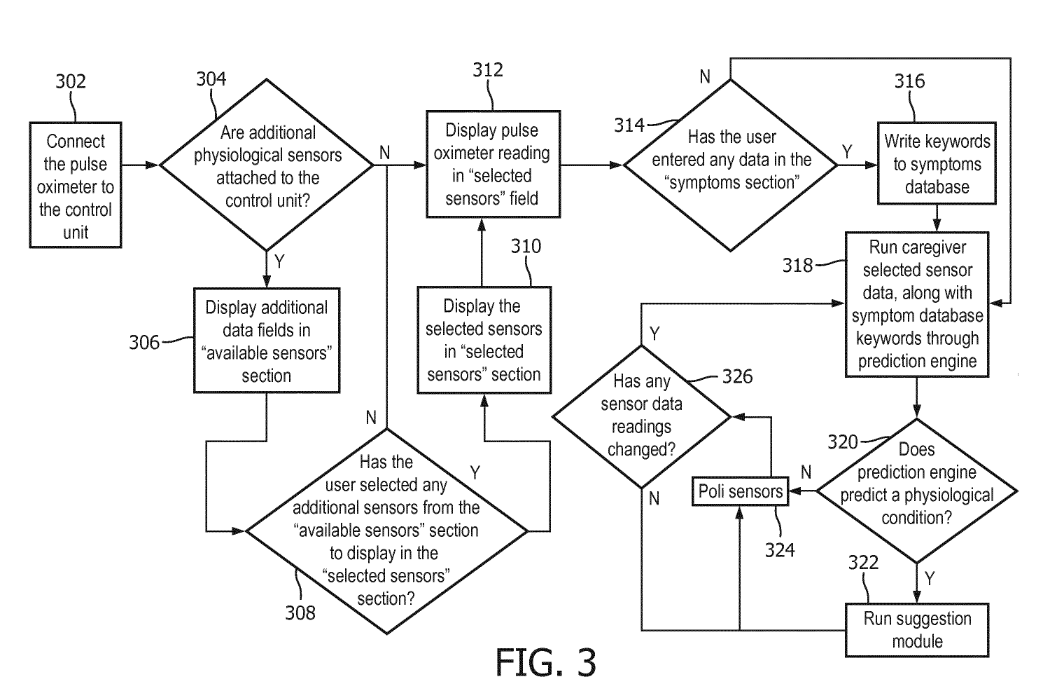

FIG. 3 illustrates a flow diagram of the steps performed by the display software 108 in accordance with an embodiment of the present invention. Initially, the pulse oximeter 102 is connected to the control unit 106 (step 302). The display software 108 then determines whether there are other physiological sensors connected to the control unit 106 (step 304). If there is at least one connected physiological sensors, the connected at least one physiological sensors are displayed in the available sensors section 126 (step 306). The display software 108 also determines whether the user selected at least one entry from the available sensor section 126 (step 308). If there is, the selected at least one entry is displayed in the sensor data section 122 (step 310). Whether or not there are additional physiological sensors attached and whether or not additional data are selected, pulse oximeter data are displayed in the sensor data section 122 (step 312).

As shown in FIG. 3, after the display of sensor data, the display software 108 determines whether there are entries inputted by the user (e.g., caregiver) in the symptoms section 124 of the patient monitor 120 (step 314). If entries have been made, these entries are added to the symptoms database 110 (step 316). All data displayed in the sensor data section 122 and inputted in the symptoms section 124 are uploaded to the cloud-based prediction engine 116. The cloud-based prediction engine 116 processes these inputs and suggests a physiological condition (step 318). The operation then proceeds to determine whether a physiological condition has been predicted (step 320). If so, a suggestion module is executed (step 322). Next, the selected sensors are polled again (step 324) to determine whether there are new data that are acquired (step 326). If so, the process loops to step 318 where the new data are again uploaded to the cloud-based prediction engine.

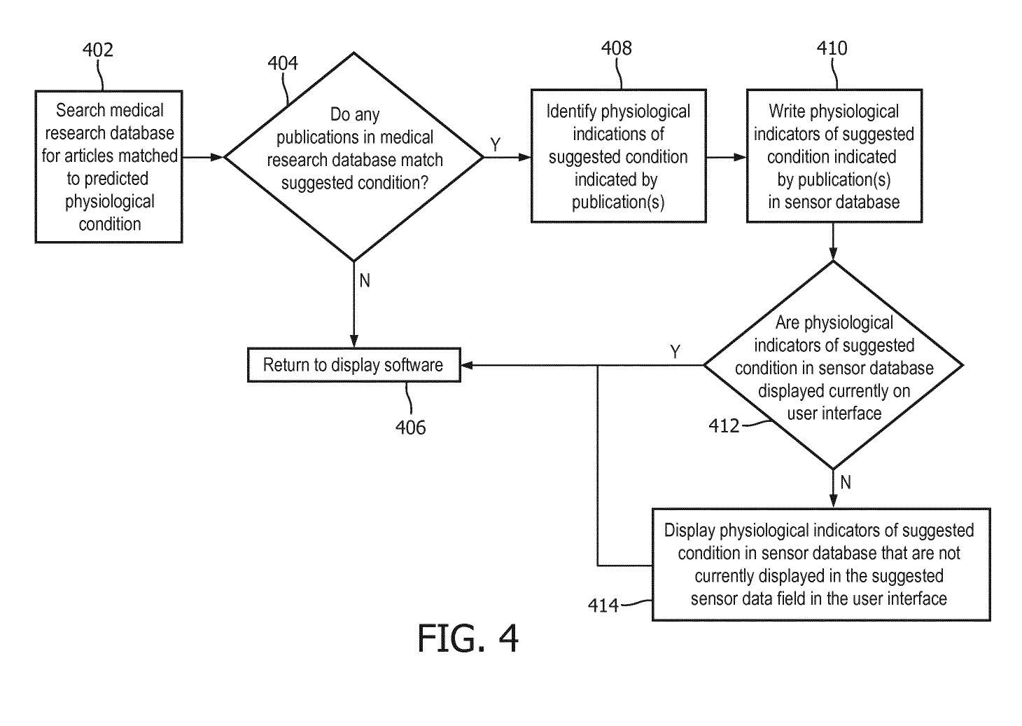

The exemplary steps followed by suggestion module is illustrated in FIG. 4. The suggestion module in accordance with an embodiment of the present invention is a software for searching online data for physiological indicators associated with a physiological condition. The suggestion module of the present invention may reside as a sub-process of the display software 108 or of the cloud-based prediction engine 115. The process followed by the suggestion module begins by searching the predicted physiological condition in a medical research database (step 402). A determination is then made on whether a match is found (step 404). If no match is found, the suggestion module terminates and returns to the display software 108 (step 406). However, if a match is found, physiological indicators are identified for the suggested physiological condition (step 408). These physiological indicators are then added to the sensors database 112 (step 410). The process then proceeds to determine whether all the physiological indicators are listed in the sensor data section 122 (step 412). Physiological indicators that are not in the sensor data section 122 are then displayed in the suggested sensors section 128 (step 414).

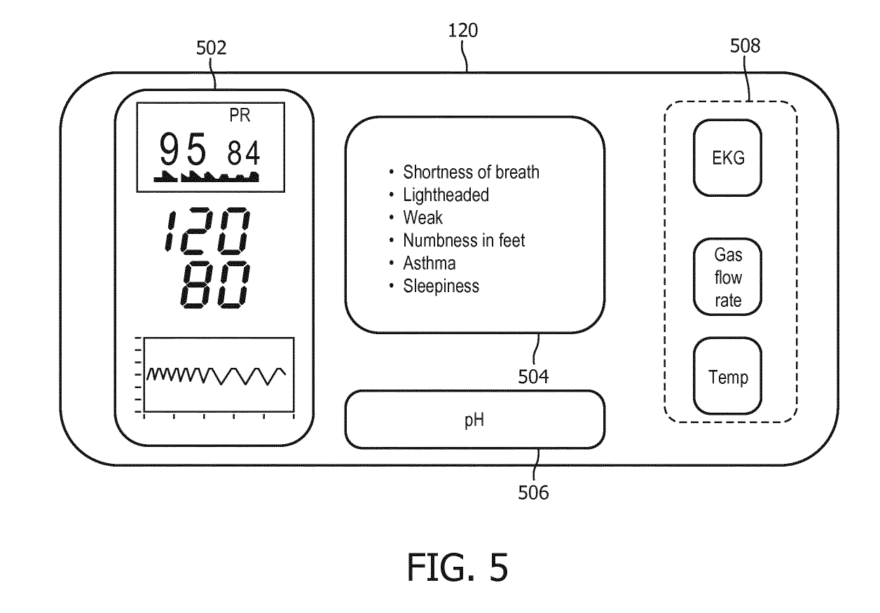

FIG. 5 illustrates various sections displayed on the patient monitor 120 in accordance with some embodiments of the present invention. The patient monitor 120 displays four sections corresponding to the sensor data section 502, symptoms and conditions section 504, suggested sensor section 506, and available sensor section 508. In this exemplary embodiment, the pulse oximeter 102 is connected to the patient and to the control unit 106. The control unit 106 displays the pulse oximeter data in the sensor data section 502. Here, SpO₂, pulse rate, systolic blood pressure, and diastolic blood pressure data are displayed. A user such as a medical professional can input symptoms and conditions in the symptom and condition section 504. For example, the medical professional inputs “shortness of breath,” “lightheaded,” “weak,” “numbness in feet,” “asthma,” and “sleepiness.” These symptoms and conditions are added to the symptom database 110. Also displayed are additional sensor data, e.g., EKG, gas flow rate, pH, from physiological sensors connected to the control unit. A medical professional can select at least one of the additional sensor data in the available sensor section 508 to be displayed in the sensor data section 502. Here, the medical professional selected the gas flow rate to display the breathing rate in the sensor data section 502. Afterwards, the data displayed on the sensor data section 502 and the entries in the symptoms and conditions section 504 are uploaded to a prediction engine to predict a physiological condition and determine whether a key indicator is not in the sensor data section 502. It is determined that the blood pH, for example, of the patient is relevant to the physiological condition predicted. The operation then proceeds to display “pH” in the suggested sensor data section 506. In response, the medical professional connects a pH meter to the patient.

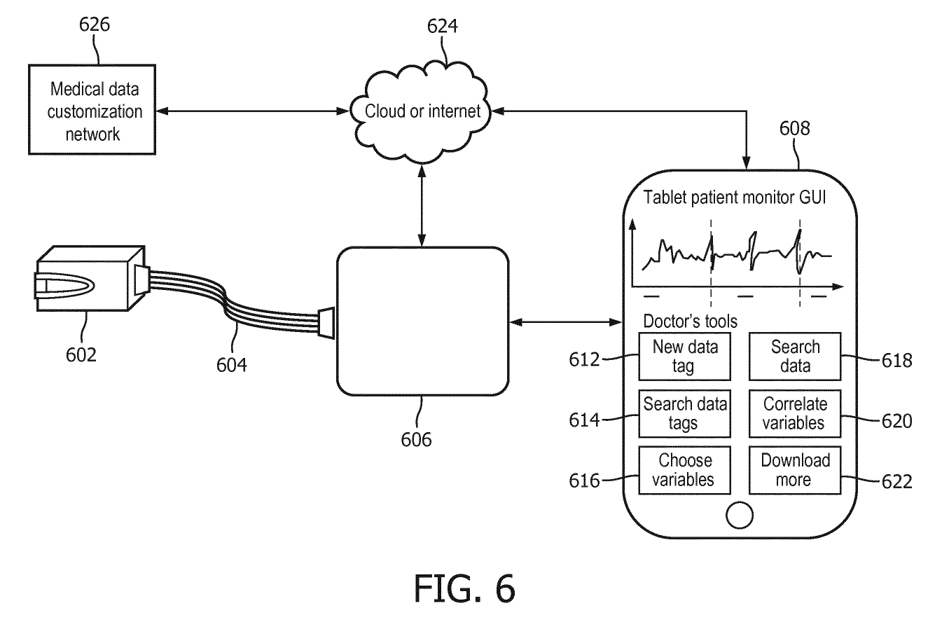

FIG. 6 illustrates another system according to an exemplary embodiment of the present invention. A pulse oximeter 602 is connected, e.g., via an integrated cable connector 604 to a control unit 606. The control unit can also be wirelessly connected to a patient monitor 608 with a graphical user interface (GUI) for analyzing and customizing data. Using the patient monitor 608, the user is presented with a GUI home screen 610 with a plurality of buttons for various analysis and customization tools, such as “New Data Tag” button 612, “Search Data Tags” button 614, “Choose Variables” button 616, “Search Data” button 618, “Correlate Variables” button 620, and “Download More” button 622. The control unit 606 and the patient monitor 608 are capable of connecting to the internet 624 and access a medical data customization network 626 for downloading and updating data analysis and customization tools.

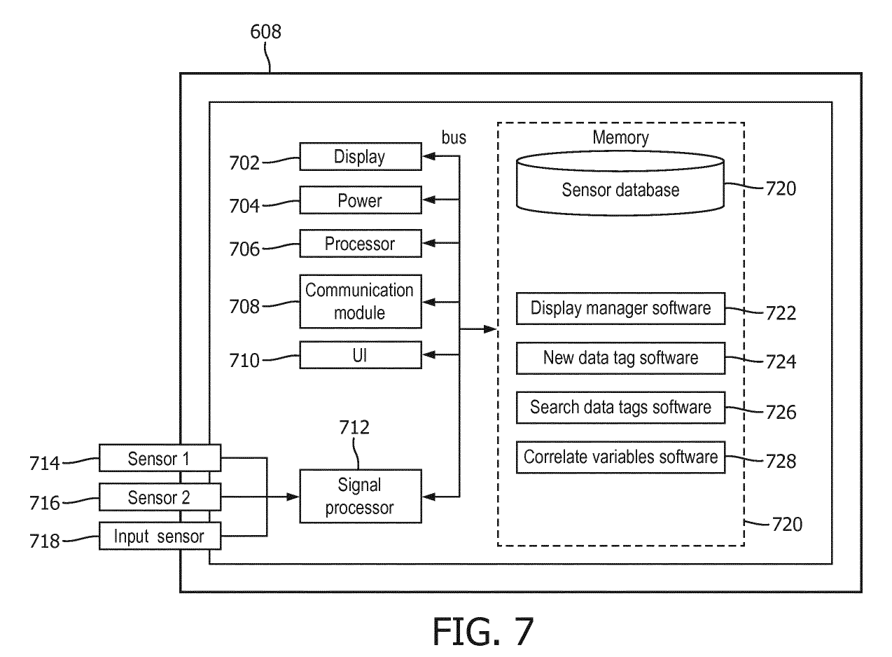

FIG. 7 illustrates an exemplary block diagram of the components of the patient monitor 608. The patient monitor 608 comprises a display module 702, a power source 704, a processor 706, a communication module 708, a user interface 710, a signal processor 712 for accepting and processing input from a plurality of sensors 714, 716 and 718, and a memory module 720 having a sensor database 722, a display manager software 724, a new data tag software 726, a search data tag software 728, and a correlate variables software 730.

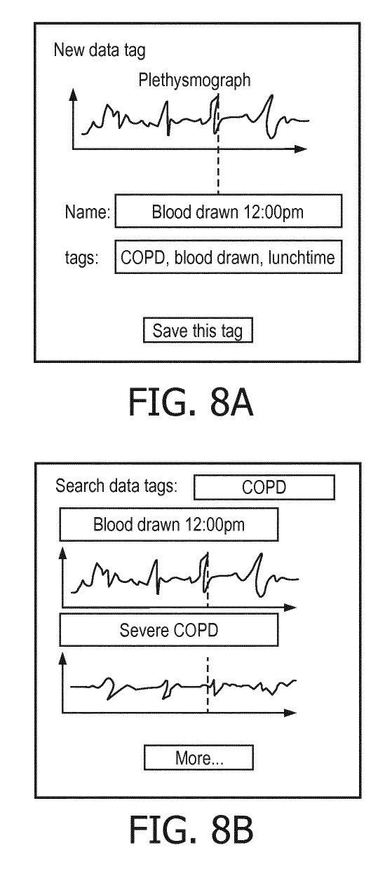

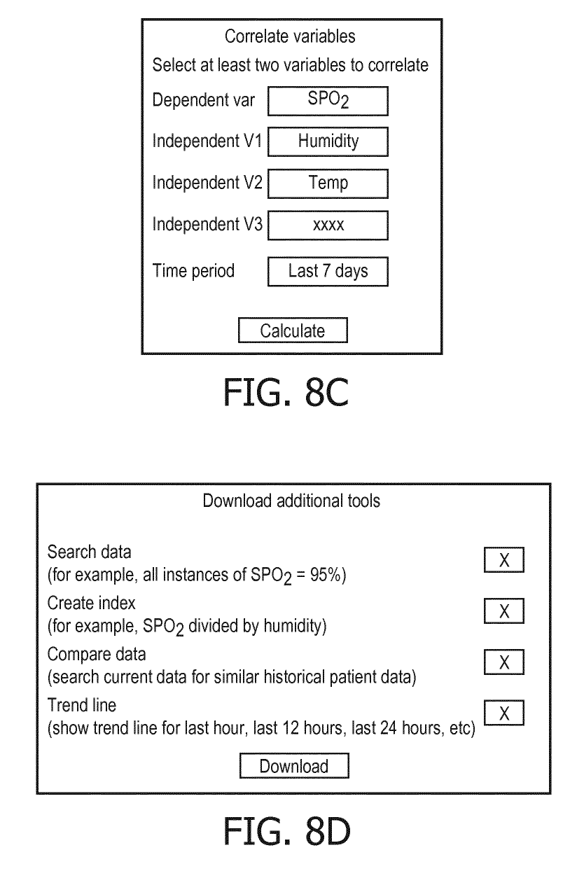

FIGS. 8A-8D are exemplary display GUIs of the patient monitor 608. In FIG. 8A is a new data tag GUI for allowing the user to select a data point on the plethysmograph and add a name and at least one tag. For example, the position indicated by the dashed line in FIG. 8A is named “Blood drawn 12:00 pm” and tagged with “COPD,” “Blood draw,” and “lunchtime.” FIG. 8B illustrates a GUI for searching data tags. In the example displayed in FIG. 8B, the user searches for chronic obstructive pulmonary disease and types in “COPD.” Consequently, all data tagged with “COPD” are displayed. FIG. 8C illustrates a GUI for correlating at least two variables. In this example, the user can select dependent variables (e.g., SpO₂), multiple independent variables (e.g., humidity and temperature) and the time period that will be considered for correlation. Upon pressing the “Calculate” button the selected data are correlated and displayed. FIG. 8D illustrates a GUI for downloading additional analytics and customization tools from the medical data customization network.

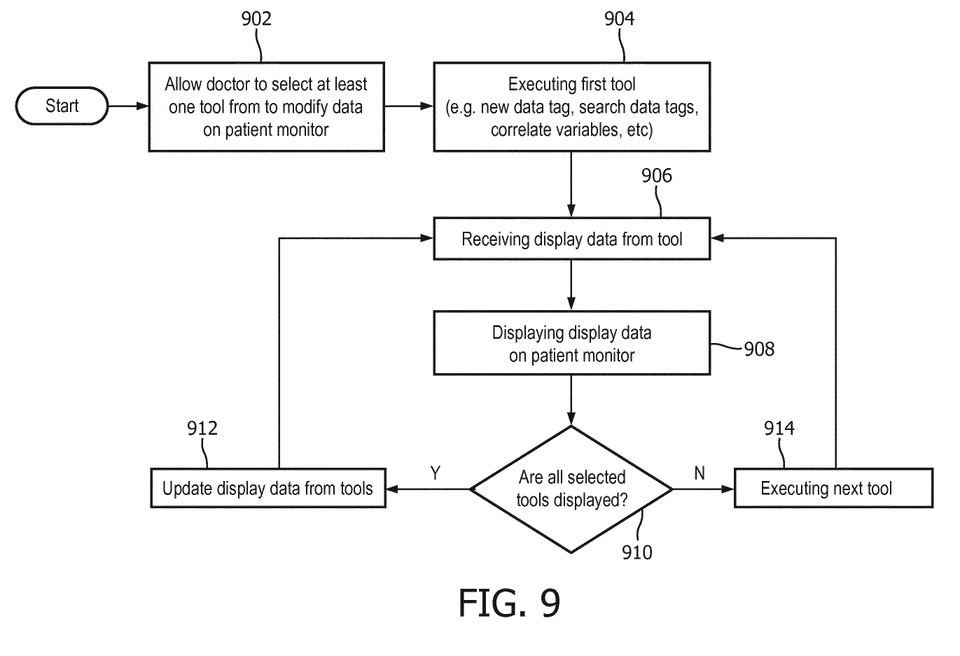

FIG. 9 shows an exemplary flow diagram of the display manager software 724 according to an embodiment of the present invention. The display manager software 724 begins by allowing a user to select at least one tool, such as a data analysis and customization tool (step 902). The operation then proceeds to execute the first tool selected (step 904). Then, the display data are retrieved by the display manager software from the first tool (step 906) and displayed (step 908). Afterwards, the display manager software 724 then determines whether all the selected tools have been executed and whether all the data from the tools have been displayed (step 910). If all the data from the selected tools are already displayed, the operation updates the data (step 912) and loops to step 906. Otherwise, the next tool is executed (step 914) and the operation loops to step 906.

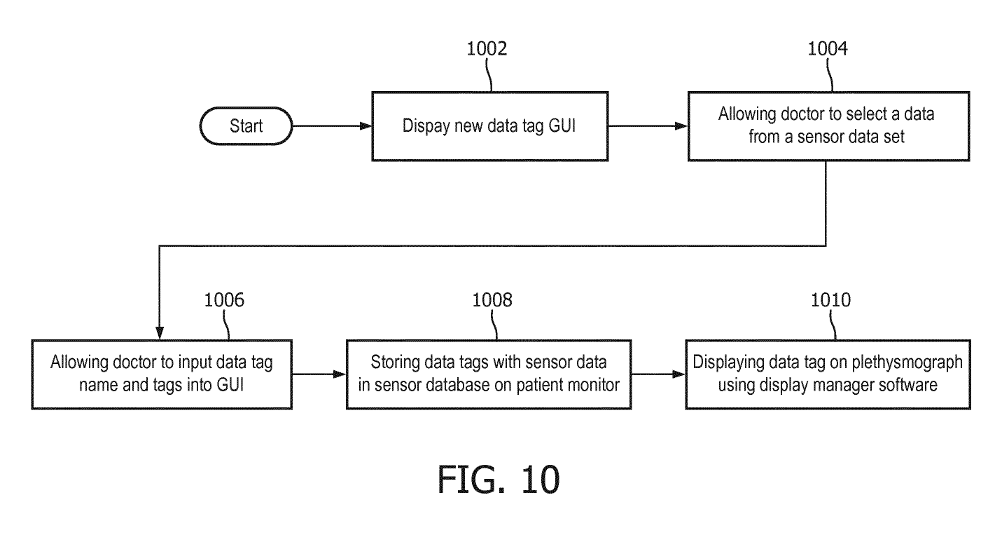

Shown in FIG. 10 is an exemplary flow diagram of the new data tag software 726 in accordance with an embodiment of the present invention. The operation of the new data tag software 726 starts with the display of the New Data Tag GUI such as in FIG. 8A (step 1002). A user (e.g., doctor) then selects at least one data from a sensor data set, such as a plethysmograph (step 1004). The user then proceeds to enter a name and at least one data tag corresponding to the selected at least one data (step 1006). These entries are saved on the sensor database 722 along with the corresponding at least one data (step 1008). The operation then return to the display manager software 724 to display the new data tags (step 1010).

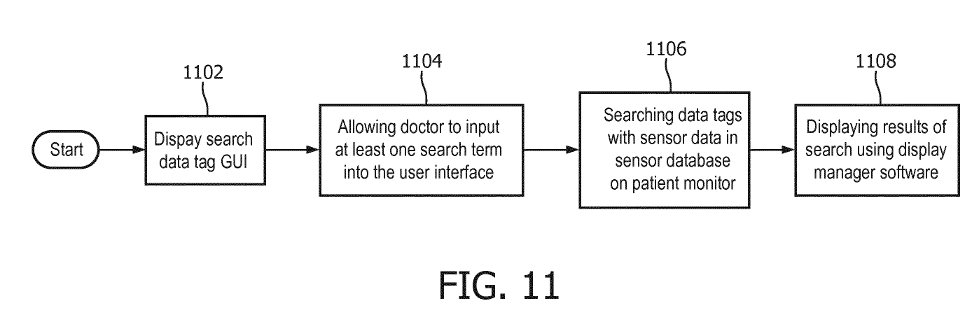

FIG. 11 shows an exemplary flow diagram of the search data tag software 728 according to an embodiment of the present invention. The operation begins with the display of Search Data Tags GUI such as in FIG. 8B (step 1102). The user then inputs at least one search term in the search field (step 1104). A search is then initiated to match the entered at least one search term to an entry in the sensor database (step 1106). The results of the search are then sent to the display manager software 724 for subsequent display (step 1108).

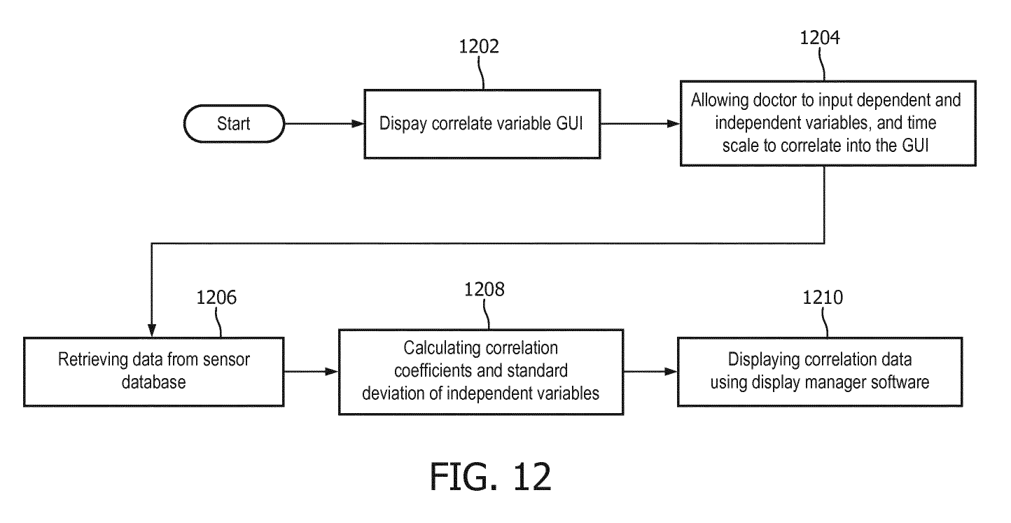

FIG. 12 is an exemplary flow diagram of the correlate variable software 730. The correlate variable GUI such as in FIG. 8C is displayed (step 1202), and the user inputs a combination of dependent and independent variables and the time period of interest (step 1204). Sensor data corresponding to the entered dependent and independent variables and time period are retrieved from the sensor database 722 (step 1206). Next, a statistical analysis is performed to calculate the correlation coefficients and standard deviations of the selected variables (step 1208). The results of the calculation are then sent to the display manager software 724 for subsequent display (step 1210).

Other data analysis and customization software are also envisioned within the scope of the present invention. Environmental data may be overlaid and correlated with physiological (sensor) data to determine their relationships. These environmental data may include ambient temperature, relative humidity, bed position, and illumination. For example, a medical professional may overlay pulse oximeter data with the relative humidity to determine what, if any, effect relative humidity has on the patient's blood oxygen saturation levels.

The present invention is not intended to be restricted to the several exemplary embodiments of the invention described above. Other variations that may be envisioned by those skilled in the art are intended to fall within the disclosure.

1. A method for predicting the condition of a patient and customizing displayed physiological data, the method comprising:
acquiring a pulse oximeter data of the patient using a first sensor, and second physiological sensor data of the patient using a second sensor, wherein the first sensor is a pulse oximeter;
uploading one or more symptoms, the acquired pulse oximeter data, and the acquired second physiological sensor data to a cloud-based prediction engine;
predicting, using the cloud-based prediction engine, a physiological condition by matching the uploaded symptoms, the acquired pulse oximeter data, and the acquired second physiological sensor data to a cloud database;
identifying one or more physiological indicators associated with the predicted physiological condition;
suggesting measuring a third physiological sensor data associated with the predicted physiological condition, the third physiological sensor data not previously uploaded by the user;
acquiring from the patient the third physiological sensor data associated with the predicted physiological condition;
displaying the acquired pulse oximeter data, the acquired second physiological sensor data, the predicted physiological condition, and the third physiological sensor data on a patient monitor; and
customizing the displayed data using a data analysis and customization tool.

2. The method of claim 1, wherein predicting includes searching a medical research database for an article that matches with the predicted physiological condition.

3. The method of claim 1, further comprising adding the identified physiological indicators to a sensor database.

4. The method of claim 3, wherein suggesting includes displaying the physiological indicators that are not listed in the sensor database on the patient monitor.

5. The method of claim 1, wherein the symptoms are selected from a shortness of breath, a lightheaded-ness, a weakness, a numbness in foot, an asthma, a sleepiness, and a combination thereof.

6. The method of claim 1, wherein the acquired second physiological sensor data is selected from an EKG, a gas flow rate, a pH, and a combination thereof.

7. A system for predicting the condition of a patient and customizing displayed physiological data using the method of claim 1, the system comprising:
a pulse oximeter;
a control unit connected to the pulse oximeter and to the internet that accesses a prediction engine and a medical research database via the internet, the control unit having a display software, a symptoms database, and a sensor database, and the control unit enabled to receive and store sensor data from a plurality of sensors including a second physiological sensor and a third physiological sensor; and
a patient monitor coupled with the control unit and having a display, a user interface, a processor, a communication modules and a memory module having one or more data analysis and customization tools.

8. The system of claim 7, wherein the prediction engine receives data uploaded by a user and uses the data together with data from another source to determine a likely physiological condition of the patient.

9. The system of claim 8, wherein data from another source is selected from a historical data from the user, a statistical data from a population of patients, a published medical data, or a combination thereof.

10. The system of claim 7, wherein the patient monitor includes a graphical user interface (GUI) for analyzing and customizing data, the GUI including a GUI home screen having a plurality of buttons related to the one or more data analysis and customization tools.

11. The system of claim 7, wherein the one or more data analysis and customization tools is selected from a display manager software, a new data tag software, a search data tag software, a correlate variables software, and a combination thereof.

12. The system of claim 11, wherein the display manager software, wherein further execution of instructions by the processor:
allows the user to select at least one of the one or more data analysis and customization tools to modify data on the patient monitor;
retrieves a display data from the selected data analysis and customization tools;
determines that all of the display data from the selected data analysis and customization tools have been displayed; and
updates the display data.

13. The system of claim 11, wherein the new data tag software, wherein further execution of instructions by the processor:
allows the user to select at least one sensor data;
receives a name and at least one data tag corresponding to the selected sensor data, entered by the user; and
stores the name and the data tag along with the corresponding sensor data on the sensor database.

14. The system of claim 11, wherein the search data tag software, wherein further execution of instructions by the processor:
allows the user to input at least one search term in a search field of the GUI;
initiates a search to match the entered search term to an entry in the sensor database; and
sends a result of the search to the display manager software.

15. The system of claim 11, wherein the correlate variable software, wherein further execution of instructions by the processor:
allows the user to input a dependent variable, an independent variable, and a time period;
retrieves sensor data that corresponds to the dependent variable, the independent variable, and the time period from the sensor database;
calculates a correlation coefficient and a standard deviation of the inputted dependent variable and the independent variable; and
sending the calculated correlation coefficient and the calculated standard deviation to the display manager software.

16. (canceled)

17. (canceled)
